(19) United States
(12) Patent Application Publication
(10) Pub. No.: US 2022/0370640 A1
(43) Pub. Date: Nov. 24, 2022

(54) GENE THERAPY EXPRESSION SYSTEM ALLEVIATING CARDIAC TOXICITY OF FKRP

(71) Applicants: GENETHON, Evry-Courcouronnes (FR); UNIVERSITE D'EVRY-VAL-D'ESSONNE, Evry-Courcouronnes (FR)

(72) Inventors: Evelyne GICQUEL, Vert-le-Petit (FR); Isabelle RICHARD, Corbeil Essonnes (FR)

(21) Appl. No.: 17/753,768

(22) PCT Filed: Sep. 18, 2020
(86) PCT No.: PCT/EP2020/076063

§ 371 Date: Mar. 14, 2022

(30) Foreign Application Priority Data
Sep. 19, 2019 (EP) 19306134.8

(51) Int. Cl. A61K 48/00; A61K 9/00; A61P 21/00

(52) U.S. Cl. CPC A61K 48/0058; A61K 48/0008; A61K 9/0019; A61P 21/00

(57) ABSTRACT

The present invention concerns an expression system for systemic administration comprising a sequence encoding a FKRP protein, and: —a promoter sequence allowing the expression at a therapeutically acceptable level of FKRP in the skeletal muscles and a target sequence of an miRNA expressed in the heart; or—a promoter sequence allowing the expression at a therapeutically acceptable level of FKRP in the skeletal muscles and presenting a promoter activity at a toxically acceptable level in the heart; and its use for the treatment of various diseases linked to FKRP deficiencies.

The present invention is based on the identification of the cardiac toxicity of FKRP (Fukutin-Related Protein) transgenic expression. It provides an expression system for alleviating FKRP toxicity in the heart, especially by modulating, i.e. partially detargeting FKRP cardiac expression. It then offers a valuable and safe therapeutic tool for the treatment of various diseases linked to FKRP deficiencies, such as Limb-Girdle Muscular Dystrophy type 2I (LGMD2I), newly named Limb girdle muscular dystrophy type R9 (LGMD2 R9).

### BACKGROUND OF THE INVENTION

The “Dystroglycanopathies” regroup different genetic pathologies leading to secondary aberrant glycosylation of α-dystroglycan (αDG). This protein, mostly present in skeletal muscle, heart, eye and brain tissues, is a hyper-glycosylated membrane protein, the glycosylation process raising its weight from 70 to 156 kDa in muscle. It is part of the dystrophin-glycoprotein complex which connects the cytoskeleton to the extracellular matrix (ECM). Its high glycosylation level enables αDG direct binding to the laminin globular domains of some ECM proteins, such as laminin in the cardiac and skeletal muscles, agrin and perlecan at the neuromuscular junction, neurexin in brain and pikachurin in the retina. Glycosylation of αDG is a complex process that is not yet fully understood. Indeed, a number of genes have been identified as being involved in αDG glycosylation. These discoveries have been accelerating recently thanks to the use of high throughput sequencing methods for mutation detection in patients showing αDG glycosylation defects. One of these proteins is the Fukutin-Related Protein (FKRP). It was originally classified as a putative αDG glycosyltransferase on account of the presence in its sequence of a D×D motif, which is common to many glycosyltransferases, and evidence of αDG hypoglycosylation in patients mutated in the FKRP gene (Breton et al., 1999; Brockington et al., 2001). Recently, FKRP and its homolog fukutin were identified as ribitol-5-phosphate (Rbo5P) transferases, forming a di-Rbo5P linker necessary for addition of the ligand binding moiety (Kanagawa et al., 2016).

Mutations in the FKRP gene can generate the entire range of pathologies induced by a defect in αDG glycosylation, from Limb-Girdle Muscular Dystrophy type 2I (LGMD2I; Muller et al., 2005; New name: Limb girdle muscular dystrophy type R9 or LGMD2 R9), Congenital Muscular Dystrophy type 1C (MDC1C; Brockington et al., 2001) to Walker-Warburg Syndrome (WWS) and Muscle-Eye-Brain disease (MEB; Beltran-Valero de Bernabe et al., 2004). There is an inverse correlation between the severity of the disease and the number of patients, the more severe, the rarer the patients (prevalence indicated in www.orphanet.fr: WWS (all genes): 1-9/1,000,000 and LGMD2I: 1-9/100,000). The type of pathology seems, at least partially, correlated to the nature of the FKRP mutation. In particular, the homozygous L276I mutation, replacing a leucine by an isoleucine in position 276 of the protein, is always associated with LGMD2I (Mercuri et al., 2003). LGMD2I is a recessive autosomal muscular dystrophy, affecting preferentially, albeit heterogeneously, the muscles of the shoulder and pelvic girdles. It is one of the most frequent LGMD2 in Europe, notably due to high prevalence of the L276I mutation in Northern Europe (Sveen et al., 2006). The severity of the pathology is very heterogeneous. The muscular symptoms can appear between the first to third decades, and vary from Duchenne-like disease to relatively benign courses. The heart can also be affected with consequences such as severe heart failure and death (Muller et al., 2005). Investigations using cardiac magnetic resonance imaging suggest that a very high proportion of LGMD2I patients (60-80%) can present myocardial dysfunction such as reduced ejection fraction (Wahbi et al., 2008). Interestingly, the severity of the cardiac abnormalities is not correlated to the skeletal muscle involvement. Based on a cohort of 7 patients, Rosales et al. (2011) concluded that LGMD2I generally results in mild structural and functional cardiac abnormalities, though severe dilated cardiomyopathy may occur (one patient). Petri et al. (2015) also observed that among patients with LGMD2, LVEF (Left Ventricular Ejection Fraction) decreased significantly in patients with LGMD type 2I (n=28) from 59% (15-72) to 55% (20-61), p=0.03, i.e. a 0.4 percentage drop annually, and LVEF≤50% was associated with increased mortality in this subgroup.

Gicquel et al. (Hum Mol Genet, 2017 Mar. 3. doi: 10.1093/hmg/ddx066) reported the generation of a FKRP^{L276I} mouse model in which the recombinant adeno-associated virus (rAAV2/9) transfer of the murine Fkrp gene, placed under the control of the desmin promoter and of the polyadenylation (polyA) signal of beta-hemoglobin (HBB2) gene, was evaluated. After intramuscular or intravenous delivery, improvement of the muscle pathology was observed. They obtained strong expression of FKRP, at mRNA as well as protein levels, and showed the rescue of αDG proper glycosylation and increase in laminin binding, that led to histological and functional rescue of the dystrophy. As reported in WO2019/008157, the muscular efficiency of this construct can still be improved by using a FKRP coding sequence having mutations avoiding supplementary transcripts generated from frameshift start codons.

Therefore, gene replacement therapy based on FKRP appears as a promising treatment of pathologies resulting from a FKRP deficiency. However, there is still a need of safe and efficient treatments.

In relation to gene therapy, a safe expression system is defined as one which ensures the production of a therapeutically effective amount of the protein in the target tissues, i.e. in the tissues wherein said protein is needed to cure the abnormalities linked to the deficiency of the native protein, without displaying any toxicity, especially in the essential and vital organs or tissues.

For example and in relation to neuromuscular diseases, WO2014/167253 reported that expression systems encoding myotubularin and calpain 3 have cardiac toxicity when systemically administered whereas said toxicity can be alleviated by introducing in said construct a target sequence of an miRNA expressed in the heart or by using a promoter sequence presenting a promoter activity at a toxically acceptable level or even no activity in the heart.

### BRIEF SUMMARY OF THE INVENTION

The present invention aims at alleviating or curing the devastating pathologies linked to a fukutin-related protein (FKRP) deficiency such as Limb-Girdle Muscular Dystrophy type 2I (LGMD2I), by providing an expression system which ensures the production of a therapeutically effective amount of the protein in the target tissues mainly the skeletal tissues and a toxically acceptable amount of the protein in the heart.

Indeed, the inventors have detected a potential cardiac toxicity of the expression system encoding FKRP. This was not expected since patients having pathologies linked to a fukutin-related protein (FKRP) deficiency such as Limb-Girdle Muscular Dystrophy type 2I (LGMD2I) often also display cardiac abnormalities. Therefore and according to the common knowledge, a sustained level of FKRP expression in the heart was considered beneficial, especially to alleviate the cardiac symptoms of FKRP-associated diseases.

It is to be noted that document WO2014/167253 which provided a list of candidate genes and associated pathologies is fully silent concerning FKRP. On another hand, document WO2016/138387 merely mentioned a putative hepatic toxicity of FKRP and the possible use of a mir122 target sequence in the expression system in order to reduce expression in the liver. Finally, document WO2019/008157 discloses the possibility to add miRNA target sequences to inhibit expression in tissues in which expression is not desired or even toxic but discourages to detarget the heart.

### Definitions

Unless otherwise defined, all technical and scientific terms used therein have the same meaning as commonly understood by one of ordinary skill in the art. The terminology used in the description is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” or “approximately” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “nucleotide sequence encoding an amino acid sequence” includes all nucleotide sequences that are degenerate versions of each other and that encode the same amino acid sequence. The phrase nucleotide sequence that encodes a protein or a RNA or a cDNA may also include introns to the extent that the nucleotide sequence encoding the protein may in some version contain an intron(s).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A protein may be “altered” and contain deletions, insertions, or substitutions of amino acid residues which produce a silent change and result in a functionally equivalent. Deliberate amino acid substitutions may be made on the basis of similarity in polarity, charge, solubility, hydrophobicity, hydrophilicity, and/or the amphipathic nature of the residues as long as the biological activity is retained. For example, negatively charged amino acids may include aspartic acid and glutamic acid; positively amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

A “variant”, as used herein, refers to an amino acid sequence that is altered by one or more amino acids. The variant may have “conservative” changes, wherein a substituted amino acid has similar structural or chemical properties, e. g. replacement of leucine with isoleucine. A variant may also have “non-conservative” changes, e. g. replacement of a glycine with a tryptophan. Analogous minor variations may also include amino acid deletions or insertions, or both. Guidance in determining which amino acid residues may be substituted, inserted, or deleted without abolishing biological or immunological activity may be found using computer programs well known in the art.

“Identical” or “homologous” refers to the sequence identity or sequence similarity between two polypeptides or between two nucleic acid molecules. When a position in both of the two compared sequences is occupied by the same base or amino acid monomer subunit, e.g., if a position in each of two DNA molecules is occupied by adenine, then the molecules are homologous or identical at that position. The percent of homology/identity between two sequences is a function of the number of matching positions shared by the two sequences divided by the number of positions compared×100. For example, if 6 of 10 of the positions in two sequences are matched then the two sequences are 60% identical. Generally, a comparison is made when two sequences are aligned to give maximum homology/identity.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “promoter” as used herein is defined as a DNA sequence recognized by the transcriptional machinery of the cell, or introduced transcriptional machinery, required to initiate the specific transcription of a polynucleotide sequence.

As used herein, the term “promoter/regulatory sequence” means a nucleic acid sequence, which is required for expression of a gene product operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, be one, which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell preferentially if the cell is a cell of the tissue type corresponding to the promoter.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics, which are normal or expected for one cell or tissue type, might be abnormal for a different cell or tissue type.

The terms “patient,” “subject,” “individual,” and the like are used interchangeably herein, and refer to any animal, or cells thereof whether in vitro or in situ, amenable to the methods described herein. A subject can be a mammal, e.g. a human, a dog, but also a mouse, a rat or a nonhuman primate. In certain non-limiting embodiments, the patient, subject or individual is a human.

A “disease” or a “pathology” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

A disease or disorder is “alleviated” or “ameliorated” if the severity of a symptom of the disease or disorder, the frequency with which such a symptom is experienced by a patient, or both, is reduced. This also includes halting progression of the disease or disorder.

A disease or disorder is “cured” if the severity of a symptom of the disease or disorder, the frequency with which such a symptom is experienced by a patient, or both, is eliminated.

A “therapeutic” treatment is a treatment administered to a subject who exhibits signs of pathology, for the purpose of diminishing or eliminating those signs. A “prophylactic” treatment is a treatment administered to a subject who does not exhibit signs of pathology or has not be diagnosed for the pathology yet, for the purpose of preventing or postponing the occurrence of those signs.

As used herein, “treating a disease or disorder” means reducing the frequency or severity of at least one sign or symptom of a disease or disorder experienced by a subject. Disease and disorder are used interchangeably herein in the context of treatment.

An “effective amount” of a compound is that amount of compound which is sufficient to provide a beneficial effect to the subject to which the compound is administered. The phrase “therapeutically effective amount”, as used herein, refers to an amount that is sufficient or effective to prevent or treat (delay or prevent the onset of, prevent the progression of, inhibit, decrease or reverse) a disease or condition, including alleviating symptoms of such diseases. An “effective amount” of a delivery vehicle is that amount sufficient to effectively bind or deliver a compound.

### DETAILED DESCRIPTION OF THE INVENTION

This invention is based on the identification by the inventors that after systemic administration, an expression system intended for the production of a FKRP protein at high level in the skeletal muscles, can simultaneously lead to an expression in the heart potentially toxic, rendering said system unsuitable for therapeutic use.

This invention provides technical solutions for this newly identified problem, particularly regarding excessive cardiac leakages besides the skeletal muscle expression of the FKRP transgene.

Thus and in general, this invention relates to an expression system comprising a sequence encoding a FKRP protein, the said expression system allowing:
the expression at a therapeutically acceptable level of the protein in the target tissue(s), advantageously in the skeletal muscles; and
the expression at a toxically acceptable level of the protein in all the tissues, especially in the heart.

In the frame of the invention, an expression system is generally defined as a polynucleotide which allows the in vivo production of FKRP. According to one aspect, said system comprises a nucleic acid encoding a FKRP protein, as well as the regulatory elements required for its expression, at least a promoter. Said expression system can then corresponds to an expression cassette. Alternatively, said expression cassette can be harboured by a vector or a plasmid. The wording “expression system” as used therein covers all aspects.

According to the invention, the target tissue is defined as the tissue or organ in which the protein is to play a therapeutic role, especially in cases where the native gene encoding this protein is defective. According to a particular embodiment of the invention, the target tissue designates the striated skeletal muscles, hereafter referred to as skeletal muscles, i.e. all the muscles involved in motor ability and the diaphragm. Other potential target tissues are the retina and the brain.

As mentioned above, the heart can also be affected in various diseases linked to FKRP deficiencies and is therefore also a potential target tissue. However and in the frame of the present application, it is shown that FKRP when overexpressed can display cardiac toxicity. Therefore and in relation to gene transfer, the expression system should be in favour of FKRP expression in the heart at a toxically acceptable level rather than at a therapeutically acceptable level since the cardiac abnormalities can be treated using different strategies, e.g. β-blockers diuretics or ACE (Angiotensin-Converting-Enzyme) inhibitors.

As demonstrated in the present application, even if FKRP can have a therapeutic role to play in the heart, its level of expression should be tightly regulated since an excess of this protein in this tissue, especially a quantity overexceeding the endogenous quantity, may prove to be harmful or even fatal, and therefore toxic.

Therefore and in the context of the invention, the heart has to be protected from this potential toxicity. According to a particular embodiment, the expression system of the invention ensures FKRP expression at a toxically acceptable level of the protein in the heart.

Thus and according to a particular aspect, the present invention relates to an expression system comprising a sequence encoding a FKRP protein, said expression system allowing:
the expression at a therapeutically acceptable level of the protein in the target tissues including the skeletal muscles and possibly the retina and the brain; and
the expression at a toxically acceptable level of the protein in all tissues, especially in the heart.

Advantageously, the present invention concerns an expression system for systemic administration comprising a sequence encoding the FKRP protein, wherein:
FKRP is expressed at a therapeutically acceptable level in the skeletal muscles; and
FKRP is expressed at a toxically acceptable level in the heart.

According to a first characteristic, the expression system of the invention comprises a sequence encoding a FKRP protein, corresponding to a transgene. In the context of the invention, the term “transgene” refers to a sequence, preferably an open reading frame, provided in trans using the expression system of the invention.

According to a particular embodiment, this sequence is a copy, identical or equivalent, of an endogenous sequence present in the genome of the body into which the expression system is introduced.

According to another particular embodiment, the endogenous sequence has one or more mutations rendering the protein partially or fully non-functional or even absent (lack of expression or activity of the endogenous protein), or not properly located in the desired subcellular compartment. In other words and preferably, the expression system of the invention is intended to be administered to a subject having a defective copy of the sequence encoding the protein and having an associated pathology. In this context, the protein encoded by the sequence carried by the expression system of the invention can therefore be defined as a protein whose mutation causes a pathology linked to a FKRP deficiency.

Thus and more generally, the sequence carried by the expression system of the invention can be defined as encoding a protein having a therapeutic activity in the context of a pathology linked to a FKRP deficiency. The concept of therapeutic activity is defined as below in connection with the term “therapeutically acceptable level”.

The sequence encoding the FKRP protein, also named ORF for “open reading frame”, is a nucleic acid sequence or a polynucleotide and may in particular be a single- or double-stranded DNA (deoxyribonucleic acid), an RNA (ribonucleic acid) or a cDNA (complementary deoxyribonucleic acid).

Advantageously, said sequence encodes a functional protein, i.e. a protein capable of ensuring its native or essential functions, especially in the skeletal muscles. This implies that the protein produced using the expression system of the invention is properly expressed and located, and is active.

According to a preferred embodiment, said sequence encodes the native protein, said protein being preferably of human origin. It may also be a derivative or a fragment of this protein, provided that the derivative or fragment retains the desired activity. Preferably, the term “derivative” or “fragment” refers to a protein sequence having at least 60%, preferably 70%, even more preferably 80% or even 90%, 95% or 99% identity with the human FKRP sequence. Proteins from another origin (non-human mammals, etc.) or truncated, or even mutated, but active proteins are for instance encompassed. Thus and in the context of the invention, the term “protein” is understood as the full-length protein regardless of its origin, as well as functional derivatives and fragments thereof.

In a particular aspect, the diseases to be treated by an expression system according to the invention are caused by mutations in at least one gene causing non-production of the FKRP protein or production of a fully or partially non-functional protein. According to the invention, the expression system helps produce this protein in an active form and in a quantity that at least partially compensates for the absence of the native protein, or another protein capable of compensating for the absence of the native protein. The administration of the expression system thus makes it possible to improve or restore a normal phenotype in the target tissue(s), particularly the skeletal muscles, in terms of mobility and breathing.

The protein of interest in the context of the present invention is advantageously FKRP of human origin (SEQ ID NO: 5), even if e.g. the murine, rat or canine versions (which sequences are available in the databases) can be used.

According to a specific embodiment, a FKRP protein is a protein consisting of or comprising the sequence shown in SEQ ID NO: 5 (corresponding to a protein of 495 aa). According to specific embodiments, FKRP is a protein having the same functions as the native human FKRP encoded by SEQ ID NO: 5, especially the ability to glycosylate α-dystroglycan (αDG) and/or to alleviate, at least partially, one or more of the symptoms associated with a defect in FKRP, especially the LGMD2I phenotype as disclosed above. It can be a fragment and/or a derivative thereof. According to one embodiment, said FKRP sequence has identity greater than or equal to 60%, 70%, 80%, 90%, 95% or even 99% with sequence SEQ ID NO: 5.

Any sequence encoding these proteins, functional therapeutical derivatives or fragments thereof, can be implemented as part of the expression system of the invention. By way of example, the corresponding nucleotide sequences (cDNA) are the sequences identified as sequence SEQ ID NO: 2, SEQ ID NO: 3, SEQ ID NO: 4, SEQ ID NO: 5, SEQ ID NO: 6, SEQ ID NO: 7 or SEQ ID NO: 8 in WO2019/008157.

According to a specific embodiment, the sequence encoding FKRP comprises or consists of nucleotides 1659 to 3146 of sequence SEQ ID NO: 1, SEQ ID NO: 3, or SEQ ID NO: 4.

Mutations in the FKRP gene, in a known manner, can generate the entire range of pathologies induced by a defect in αDG glycosylation, from Limb-Girdle Muscular Dystrophy type 2I (LGMD2I; Muller et al., 2005), Congenital Muscular Dystrophy type 1C (MDC1C; Brockington et al., 2001) to Walker-Warburg Syndrome (WWS) and Muscle-Eye-Brain disease (MEB; Beltran-Valero de Bernabe et al., 2004). Thus and according to the strategy for replacement or transfer of the gene, the provision in trans of a sequence encoding a therapeutic FKRP, which is for example native, helps to treat said pathologies.

The present invention refers to the FKRP whose mutation causes a disease in one or more target tissues, especially in the skeletal muscles, and which production from an expression system exhibits toxicity in at least one tissue, especially the heart.

According to the invention and advantageously, the expression system must allow the expression at a therapeutically acceptable level of the FKRP protein in the skeletal muscles.

Moreover and according to another preferred embodiment, it must allow the expression at a toxically acceptable level of the FKRP protein in the heart.

In the context of the invention, the term “protein expression” may be understood as “protein production”. Thus, the expression system must allow for both transcription and translation of the protein at the levels defined above. Also important is the correct folding and localisation of said protein.

The levels defined in the context of the invention, namely “therapeutically acceptable” and “toxically acceptable” are related to the amount of protein, as well as its activity.

The evaluation of the amount of protein produced in a given tissue can be carried out by immunodetection using an antibody directed against said protein, for example by Western blot or ELISA, or by mass spectrometry. Alternatively, the corresponding messenger RNAs may be quantified, for example by PCR or RT-PCR. This quantification can be performed on one sample of the tissue or on several samples. Thus and in the case where the target tissues are skeletal muscles, it may be carried out on a muscular type or several types of muscles (for example quadriceps, diaphragm, tibialis anterior, triceps, etc.).

In the context of the invention, the term “therapeutically acceptable level” refers to the fact that the protein produced from the expression system of the invention helps improve the pathological condition of the patient, particularly in terms of quality of life or lifespan. Thus and in connection with a disease affecting skeletal muscles, this involves improving the muscular condition of the subject affected by the disease or restoring a muscular phenotype similar to that of a healthy subject. As mentioned above, the muscular state, mainly defined by the strength, size, histology and function of the muscles, can be evaluated by one of the following methods: biopsy, measurement of the strength, muscle tone, volume, or mobility of muscles, clinical examination, medical imaging, biomarkers, etc.

Thus, the criteria that help assess a therapeutic benefit as regards skeletal muscles and that can be evaluated at different times after the treatment are in particular at least one among:
increased life expectancy;
increased muscle strength
improved histology; and/or
improved functionality of the diaphragm.

In the context of the invention, the term “toxically acceptable level” refers to the fact that the protein produced from the expression system of the invention does not cause significant alteration of the tissue, especially histologically, physiologically and/or functionally. In particular, the expression of the protein may not be lethal. In one particular embodiment, the amount of protein produced in said tissue must not exceed the endogenous level of said protein in this tissue, in particular compared to a healthy subject. The toxicity in a tissue can be evaluated histologically, physiologically and functionally.

In the particular case of the heart, any toxicity of a protein can be evaluated by a study of the morphology and the heart function, by clinical examination, electrophysiology, imaging, biomarkers, monitoring of the life expectancy or by histological analysis, including the detection of fibrosis and/or cellular infiltrates and/or inflammation, for example by staining with sinus red or hematoxyline (e.g. Hematoxyline-Eosin-Saffran (HES) or Hematoxyline-Phloxin-Saffron (HFS)).

Advantageously, the level of efficacy and/or toxicity of the expression system according to the invention is evaluated in vivo in the animal, possibly in an animal having a defective copy of the gene encoding the protein and thus affected by the associated pathology. Preferably, the expression system is administered systemically, for example by intravenous (i.v.) injection.

According to the invention and preferably, the expression system comprises at least one sequence that allows to:
prevent the expression or decrease the level of expression of the protein in the tissues where the expression of the protein is toxic, especially in the heart; and/or
maintain the expression or increase the level of expression of the protein in the target tissue(s), especially in the skeletal muscles and possibly in the retina and/or in the brain.

According to a particular embodiment, the invention relates to an expression system wherein it comprises at least one sequence:
preventing the expression or reducing the level of expression of FKRP in the heart; and/or
maintaining the expression or increasing the level of expression of FKRP in the skeletal muscles.

In the context of the invention, the terminology “prevent the expression” preferably refers to cases where, even in the absence of the said sequence, there is no expression, while the terminology “decrease the level of expression” refers to cases where the expression is decreased (or reduced) by the provision of said sequence.

Similarly, the terminology “maintain the expression” preferably refers to cases where, even in the absence of said sequence, there is a comparable level of expression, while the terminology “increase the level of expression” refers to cases where there is an increase in expression by the provision of said sequence.

In the context of the invention, there are at least three ways, which may be combined, to achieve the desired objective:
using a sequence capable of preventing the expression or reducing the level of expression of the protein in the tissues where it is toxic, without reducing the level of expression in the target tissue(s);
the use of a promoter sequence capable of ensuring a high level of expression in the target tissue(s) and low or no expression in the tissues where the expression of the protein appears toxic;
the use of a vector, preferably viral, having a suitable tropism, i.e. higher for the target tissue(s) than for the tissues where the expression of the protein appears toxic.

According to one aspect, the present inventions concerns an expression system for systemic administration comprising a sequence encoding a FKRP protein, and:
a promoter sequence allowing the expression at a therapeutically acceptable level of FKRP in the skeletal muscles and a target sequence of an miRNA expressed in the heart; or
a promoter sequence allowing the expression at a therapeutically acceptable level of FKRP in the skeletal muscles and presenting a promoter activity at a toxically acceptable level in the heart.

Suitably, an expression system of the invention comprises a promoter sequence governing the transcription of the sequence encoding the protein, preferably placed at 5′ of the transgene and functionally linked thereto. Preferably, this ensures a therapeutically acceptable level of expression of the protein in the skeletal muscles.

This may include inducible or constitutive, natural or synthetic (artificial) promoters. Similarly, they can be of any origin, including human, of the same origin as the transgene or of another origin.

According to a first embodiment, the promoter sequence corresponds to a non-selective promoter, that is to say a promoter with low tissue specificity and ensuring a broadly similar level of expression in different tissues, possibly in the skeletal muscles and in the heart. The following can be cited as examples: the cytomegalovirus (CMV), phosphoglycerate kinase 1 (PGK), EF1, or CMV early enhancer/chicken β-actin (CAG) promoter.

According to a particular embodiment, this refers to a promoter sequence suitable for skeletal muscle expression but which can lead expression in other tissues, especially in other muscles, e.g. in the heart. Such promoters are considered to be muscle-specific but they are not muscle-exclusive. The following can be cited as an example: the promoter sequences coming from the desmin promoter, preferably of sequence SEQ ID NO: 6, the skeletal alpha-actin promoter (ACTA1), the muscle creatine kinase (MCK) promoter or the myosin heavy chain promoter and their derivatives such as the CK4 and MHCK7 promoters, or the C5-12 synthetic promoter.

According to a preferred embodiment of the invention, the promoter sequence of the expression system is chosen for its different promoter activity in the different tissues. In this case, this sequence helps increase the expression of the protein in the skeletal muscles, while preventing expression in the tissues in which the expression of the protein is toxic, mainly in the heart.

By way of example and in the case where the target tissue is skeletal muscle, the promoter is preferably a muscle-specific promoter. According to another advantageous characteristic, said promoter has low or no promoter activity in the heart, enabling a toxically acceptable level of expression of the protein in this tissue. More advantageously, a low promoter activity in the heart is preferred.

According to a particular embodiment, said promoter sequence may correspond to a sequence from the promoter of the calpain 3 gene, preferably of human origin, even more preferably of sequence SEQ ID NO: 7. Another suitable promoter sequence is that of the miRNA 206 (miR206), preferably of human origin, more preferably of sequence SEQ ID NO: 8. These 2 promoters have been reported in document WO2014/167253 to be capable of ensuring the expression at a therapeutically acceptable level of calpain 3 in the skeletal muscles, and at a toxically acceptable level of said protein in the heart.

According to a specific embodiment, the present invention therefore relates to an expression system comprising a sequence encoding a FKRP protein, placed under the control of a promoter having the sequence SEQ ID NO: 7 or SEQ ID NO: 8. Promoter sequences derived from the sequences SEQ ID NO: 7 and SEQ ID NO: 8 or corresponding to a fragment thereof but having a similar promoter activity, particularly in terms of tissue specificity and optionally effectiveness, are also covered under the present invention.

Any promoter displaying the above-defined expression profile, advantageously very low in heart but sufficient or even very strong in skeletal muscle, may be used.

Candidate promoter sequences can be derived from genes for which a high activity in the skeletal muscles has been reported and possibly with the desired expression profile, for example:
The promoter of the gamma-sarcoglycan gene;
The skeletal alpha-actin (ACTA1) promoter or derived versions thereof;
A Muscle Hybrid (MH) promoter as disclosed by Piekarowicz et al. (2017, European Society Of Gene & Cell Therapy conference, poster P096; HUMAN GENE THERAPY 28:A44 (2017), DOI: 10.1089/hum.2017.29055.abstracts);
Derivatives of the muscle creatine kinase promoter, especially a truncated MCK promoter with double (dMCK) or triple (tMCK) tandem of MCK enhancer, or the CK6 and CK8 promoters, as disclosed by Hauser et al. (2000, Molecular Therapy, Vol. 2, No 1, pages 16-24) and Wang et al. (2008, Gene Therapy, Vol. 15, pages 1489-99);
Promoters containing at least one sequence USE (UpStream Enhancer) as e.g. identified in the troponin I promoter sequence (Corin et al., 1995, Proc. Natl. Acad. Sci., Vol. 92, pages 6185-89), or a 100-bp deletion thereof (AUSE; Blain et al., 2010, Human Gene Therapy, Vol. 21, pages 127-34), possibly in 3 (×3) or 4 (×4) copies. Of particular interest are the DeltaUSEx3 (DUSEx3) promoter and the DeltaUSEx4 (DUSEx4) promoter.

Promoters of other genes can be further mentioned: troponin, myogenic factor 5 (Myf5), myosin light chain 1/3 fast (MLC1/3f), myogenic differentiation 1 (MyoD1), myogenin (Myog), paired box gene 7 (Pax7), MEF2.

Promoter sequences derived from said sequences or corresponding to a fragment thereof but having a similar promoter activity, particularly in terms of tissue specificity and possibly effectiveness, are also covered under the present invention. Preferably, the term “derivative” or “fragment” refers to a sequence having at least 60%, preferably 70%, even more preferably 80% or even 90%, 95% or 99% identity with said sequences. Of particular interest are the promoter sequences allowing an adequate FKRP expression in the skeletal muscles and in the heart as defined above.

According to one embodiment, the expression system of the invention comprises:
a sequence encoding the FKRP protein, and
a promoter sequence allowing the expression at a therapeutically acceptable level of FKRP in the skeletal muscles and presenting a promoter activity at a toxically acceptable level or even no activity in the heart, possibly one of these listed above.

In case this promoter sequence does not allow expression at a toxically acceptable level of the FKRP protein in all tissues, especially in the heart, it is advantageously associated with a sequence having the function of reducing the level of expression of the FKRP protein in said tissue, where the expression of the protein is toxic.

Thus, the present application reports that the use of a desmin promoter for expressing FKRP resulted in cardiac toxicity. In contrast and in accordance with the invention, the use of a desmin promoter, preferably of sequence SEQ ID NO: 6, associated with at least one target sequence of the miRNA-208a, preferably of sequence SEQ ID NO: 2, allows both: a therapeutically acceptable level of expression of the protein in the skeletal muscles;
a toxically acceptable level of expression of the protein in the heart.

As already stated, said sequence is capable of preventing the expression or reducing the level of expression of the FKRP protein in the tissues where protein expression is toxic, especially in the heart. This action may take place according to various mechanisms, particularly:
with regard to the level of transcription of the sequence encoding the protein;
with regard to transcripts resulting from the transcription of the sequence encoding the protein, e.g., via their degradation;
with regard to the translation of the transcripts into protein.

Such a sequence is preferably a target for a small RNA molecule e.g. selected from the following group:
microRNAs;
endogenous small interfering RNA or siRNAs;
small fragments of the transfer RNA (tRNA);
RNA of the intergenic regions;
Ribosomal RNA (rRNA);
Small nuclear RNA (snRNA);
Small nucleolar RNAs (snoRNA);
RNA interacting with piwi proteins (piRNA).

Advantageously, this sequence helps maintain the expression, or even increase the level of expression of the FKRP protein in the target tissue(s), preferably in the skeletal muscles.

Preferably, such a sequence is selected for its effectiveness in the tissue wherein the expression of the protein is toxic. Since the effectiveness of this sequence can be variable depending on the tissues, it may be necessary to combine several of these sequences, chosen for their effectiveness in all target tissues where toxicity is proven.

According to a preferred embodiment, this sequence is a target sequence for a microRNA (miRNA). As known, such a judiciously chosen sequence helps to specifically suppress gene expression in selected tissues.

Thus and according to a particular embodiment, the expression system of the invention comprises a target sequence for a microRNA (miRNA) expressed or present in the tissue(s) in which the expression of the protein is toxic, especially in the heart. Suitably, the quantity of this miRNA present in the target tissue, preferably the skeletal muscles, is less than that present in the tissues wherein FKRP is toxic, or this miRNA may not even be expressed in the target tissue. According to a particular embodiment, the target miRNA is not expressed in the skeletal muscles. According to another particular embodiment, it is specifically or even exclusively expressed in the heart.

As is known to the person skilled in the art, the presence or level of expression, particularly in a given tissue, of a miRNA may be assessed by PCR, preferably by RT-PCR, or by Northern blot.

Different miRNAs, as well as their target sequence and their tissue specificity, are known to those skilled in the art and are for example described in the document WO 2007/000668. MiRNAs expressed in the heart are e.g. miR-1, miR133a, miR-206, miR-499, and miR-208a. Of particular interest are the miRNAs exclusively expressed in the heart such as miR208a of sequence SEQ ID NO: 21.

According to a particular embodiment, the expression system of the invention comprises a target sequence of miRNA-208a (also noted miR208a; SEQ ID NO: 21). Thus, it has been shown within the framework of the invention that the use of such a target sequence in relation to FKRP makes it possible to solve the problem of its cardiac toxicity. Preferably, this target sequence, identical in humans, dogs and mice, has the sequence SEQ ID NO: 2 of 22 pb. Of course, any derived or truncated sequence recognised by miRNA-208a may be implemented as part of the invention. In particular, a sequence diverging from SEQ ID NO: 2 in one or several nucleotides, e.g. having at least 60%, 70%, 80%, 90% or even 95% identity with SEQ ID NO: 2, can be used as long as it is able to bind miR208a, i.e. it is a target sequence of miR208a respecting preferably the homology with its seed sequence.

As already stated, a target sequence for a microRNA may be used alone or in combination with other sequences, advantageously target sequences for a microRNA, which may be identical or different. These sequences can be used in tandem or in opposite direction. In relation to FKRP, the use of a target sequence of mir122 expressed in the liver has already been suggested.

According to a preferred embodiment, particularly for the target sequence of the miRNA208a, one (1) or more, particularly two (2) or four (4) sequences, may be implemented. Preferably, they are used in tandem, that is to say, all in the same direction. In cases where multiple target sequences are implemented, they may be separated by a DNA spacer of random sequence, in a manner known to those skilled in the art.

Preferably, in the case of a target sequence of a miRNA, particularly the miR208a, it is placed at 3′ of the sequence encoding the protein, more advantageously inserted into the 3′ UTR (“Untranslated Region”) region of the expression system. Even more preferably and when the expression system comprises a polyadenylation signal at 3′ of the cDNA encoding the protein, this sequence is inserted between the stop codon of the open reading frame and the polyadenylation signal.

In the context of the invention, it has been demonstrated that at least one target sequence of the miRNA-208a was adapted to obtain a toxically acceptable level of the FKRP protein at least in the heart.

According to one embodiment, the expression system of the invention comprises:
a sequence encoding the FKRP protein; and
a target sequence of an miRNA expressed in the heart.

Besides and preferably, it further comprises a promoter sequence which governs the expression of FKRP. Said promoter is preferably a promoter sequence allowing the expression of FKRP at a therapeutically acceptable level in the skeletal muscles, e.g. the desmin promoter preferably that of human desmin (SEQ ID NO: 6).

According to a particular embodiment, the expression system comprises:
a sequence encoding FKRP placed under the control of a promoter allowing muscle expression, e.g. that of desmin, preferably that of human desmin e.g. of sequence SEQ ID NO: 6;
at least one target sequence of a miRNA expressed in the heart, preferably of the miRNA-208a, preferably the target sequence SEQ ID NO: 2.

According to specific embodiments, an expression system according to the invention comprises or consists of:
nucleotides 146 to 3946 of SEQ ID NO: 3; or
nucleotides 146 to 3974 of SEQ ID NO: 4.

In another particular form of embodiment, the expression system may comprise:
a sequence encoding FKRP placed under the control of a promoter, e.g. that of desmin, preferably that of human desmin e.g. of sequence SEQ ID NO: 6, or that of calpain 3, preferably that of human calpain 3 e.g. of sequence SEQ ID NO: 7, or that of miRNA206, preferably that of human miRNA206 e.g. of sequence SEQ ID NO: 8;
at least one target sequence of a miRNA expressed in the heart, preferably of the miRNA-208a, e.g. of sequence SEQ ID NO: 2, possibly two target sequences advantageously in tandem.

Thus, different types of sequences detailed above may be combined in the same expression system.

According to the invention, an expression system or expression cassette comprises the elements necessary for the expression of the transgene present. In addition to sequences such as those defined above to ensure and to modulate transgene expression, such a system may include other sequences such as:
A polyadenylation signal, for example polyA of the SV40 or human haemoglobin, preferably inserted at 3′ of the coding sequence, or 3′ of the target sequence of the miRNA;
Sequences to stabilise the transcripts, such as intron 1 of human hemoglobin;
Enhancer sequences.

An expression system according to the invention can be introduced in a cell, a tissue or a body, particularly in humans. In a manner known to those skilled in the art, the introduction can be done ex vivo or in vivo, for example by transfection or transduction. According to another aspect, the present invention therefore encompasses a cell or a tissue, preferably of human origin, comprising an expression system of the invention.

The expression system according to the invention, in this case an isolated nucleic acid, can be administered in a subject, namely in the form of a naked DNA. To facilitate the introduction of this nucleic acid in the cells, it can be combined with different chemical means such as colloidal disperse systems (macromolecular complex, nanocapsules, microspheres, beads) or lipid-based systems (oil-in-water emulsions, micelles, liposomes).

Alternatively and according to another preferred embodiment, the expression system of the invention comprises a plasmid or a vector. Advantageously, such a vector is a viral vector. Viral vectors commonly used in gene therapy in mammals, including humans, are known to those skilled in the art. Such viral vectors are preferably chosen from the following list: vector derived from the herpes virus, baculovirus vector, lentiviral vector, retroviral vector, adenoviral vector and adeno-associated viral vector (AAV).

According to a specific embodiment of the invention, the viral vector containing the expression system is an adeno-associated viral (AAV) vector.

Adeno-associated viral (AAV) vectors have become powerful gene delivery tools for the treatment of various disorders. AAV vectors possess a number of features that render them ideally suited for gene therapy, including a lack of pathogenicity, moderate immunogenicity, and the ability to transduce post-mitotic cells and tissues in a stable and efficient manner. Expression of a particular gene contained within an AAV vector can be specifically targeted to one or more types of cells by choosing the appropriate combination of AAV serotype, promoter, and delivery method.

In one embodiment, the encoding sequence is contained within an AAV vector. More than 100 naturally occurring serotypes of AAV are known. Many natural variants in the AAV capsid exist, allowing identification and use of an AAV with properties specifically suited for dystrophic pathologies. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus.

As mentioned above, the use of AAV vectors is a common mode of exogenous delivery of DNA as it is relatively non-toxic, provides efficient gene transfer, and can be easily optimized for specific purposes. Among the serotypes of AAVs isolated from human or non-human primates (NHP) and well characterized, human serotype 2 is the first AAV that was developed as a gene transfer vector. Other currently used AAV serotypes include AAV1, AAV3, AAV4, AAV5, AAV6, AAV7, AAV8, AAV9, AAVrh10, AAVrh74, AAV11 and AAV12. In addition, non-natural engineered variants and chimeric AAV can also be useful.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells.

Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Thus exemplary AAVs, or artificial AAVs, include AAV2/8 (U.S. Pat. No. 7,282,199), AAV2/5 (available from the National Institutes of Health), AAV2/9 (WO2005/033321), AAV2/6 (U.S. Pat. No. 6,156,303), AAVrh10 (WO2003/042397), AAVrh74 (WO2003/123503), AAV9-rh74 hybrid or AAV9-rh74-P1 hybrid (WO2019/193119), AAV variants disclosed in PCT/EP2020/061380 among others. In one embodiment, the vectors useful in the compositions and methods described herein contain, at a minimum, sequences encoding a selected AAV serotype capsid, e.g., an AAV8 capsid, or a fragment thereof. In another embodiment, useful vectors contain, at a minimum, sequences encoding a selected AAV serotype rep protein, e.g., AAV8 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV8 origin. Alternatively, vectors may be used in which the rep sequences are from an AAV serotype, which differs from that which is providing the cap sequences.

In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 (U.S. Pat. No. 7,282,199).

According to one embodiment, the composition comprises an AAV of serotype 2, 5, 8 or 9, or an AAVrh74. Advantageously, the claimed vector is an AAV8 or AAV9 vector, especially an AAV2/8 or AAV2/9 vector. More advantageously, the claimed vector is an AAV9 vector or an AAV2/9 vector.

In the AAV vectors used in the present invention, the AAV genome may be either a single stranded (ss) nucleic acid or a double stranded (ds)/self complementary (sc) nucleic acid molecule.

Advantageously, the polynucleotide encoding the FKRP protein is inserted between the ITR («Inverted Terminal Repeat») sequences of the AAV vector. Typical ITR sequences correspond to nucleotides 1 to 145 of SEQ ID NO: 1 (5′ITR sequences) and nucleotides 3913 to 4057 of SEQ ID NO: 1 (3′ITR sequences).

Recombinant viral particles can be obtained by any method known to the one skilled in the art, e.g. by co-transfection of 293 HEK cells, by the herpes simplex virus system and by the baculovirus system. The vector titers are usually expressed as viral genomes per mL (vg/mL).

In one embodiment, the vector comprises regulatory sequences, especially a promoter sequence, advantageously as described above.

A non-exhaustive list of other possible regulatory sequences is:
sequences for transcript stabilization, e.g. intron 1 of hemoglobin (HBB2), e.g. corresponding to nucleotides 1207 to 1652 of SEQ ID NO: 1. As shown in sequence SEQ ID NO: 1, said HBB2 intron is advantageously followed by consensus Kozak sequence (GCCACC) included before AUG start codon within mRNA, to improve initiation of translation;
a polyadenylation signal, e.g. the polyA of the gene of interest, the polyA of SV40 or of beta hemoglobin (HBB2), advantageously in 3′ of the sequence encoding the human FKRP. As a preferred example, the poly A of HBB2 corresponds to nucleotides 3147 to 3912 of SEQ ID NO: 1;
enhancer sequences;
miRNA target sequences, which can inhibit the expression of the sequence encoding the human FKRP in non target tissues, in which said expression is not desired, for example where it can be toxic. As an example, it can be the target sequence of miR122 in order to avoid hepatic toxicity. Preferably, the corresponding miRNA is not present in the skeletal muscles.

In relation to a polynucleotide encoding the sequence SEQ ID NO: 5 and corresponding e.g. to nucleotides 1659 to 3146 of SEQ ID NO: 1, a vector of the invention may comprise the sequences shown in SEQ ID NO: 1, SEQ ID NO: 3, and SEQ ID NO: 4 respectively.

According to a preferred embodiment, the expression system of the invention includes a vector having a suitable tropism, in this case higher for the target tissue(s), advantageously the skeletal muscles than for the tissues where the expression of the protein appears toxic. Advantageously, the expression system of the invention includes a vector having a tropism higher for the skeletal muscles than for the heart. It can be an AAV vector containing a capsid selected for minimum or no targeting/transducing the heart or to preferentially or even exclusively target/transduce the skeletal muscles.

Further aspects of the invention concern:
A cell comprising the expression system of the invention or a vector comprising said expression system, as disclosed above.

The cell can be any type of cells, i.e. prokaryotic or eukaryotic. The cell can be used for propagation of the vector or can be further introduced (e.g. grafted) in a host or a subject. The expression system or vector can be introduced in the cell by any means known in the art, e.g. by transformation, electroporation or transfection. Vesicles derived from cells can also be used.
A transgenic animal, advantageously non-human, comprising the expression system of the invention, a vector comprising said expression system, or a cells comprising said expression system or said vector, as disclosed above.

Another aspect of the invention relates to a composition comprising an expression system, a vector or a cell, as disclosed above, for use as a medicament.

According to an embodiment, the composition comprises at least said gene therapy product (the expression system, the vector or the cell), and possibly other active molecules (other gene therapy products, chemical molecules, peptides, proteins . . . ), dedicated to the treatment of the same disease or another disease.

According to a specific embodiment, the use of the expression system according to the invention is combined with the use of anti-inflammatory drugs or ribitol.

The present invention then provides pharmaceutical compositions comprising an expression system, a vector or a cell of the invention. Such compositions comprise a therapeutically effective amount of the therapeutic (the expression system or vector or cell of the invention), and a pharmaceutically acceptable carrier. In a specific embodiment, the term “pharmaceutically acceptable” means approved by a regulatory agency of the Federal or a state government or listed in the U.S. or European Pharmacopeia or other generally recognized pharmacopeia for use in animals, and humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, sustained-release formulations and the like. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the subject.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to release pain at the site of the injection.

In one embodiment, the composition according to the invention is suitable for administration in humans. The composition is preferably in a liquid form, advantageously a saline composition, more advantageously a phosphate buffered saline (PBS) composition or a Ringer-Lactate solution.

The amount of the therapeutic (i.e. an expression system or a vector or a cell) of the invention which will be effective in the treatment of the target diseases can be determined by standard clinical techniques. In addition, in vivo and/or in vitro assays may optionally be employed to help predict optimal dosage ranges. The precise dose to be employed in the formulation will also depend on the route of administration, the weight and the seriousness of the disease, and should be decided according to the judgment of the practitioner and each patient's circumstances.

Suitable administration should allow the delivery of a therapeutically effective amount of the gene therapy product to the target tissues, especially skeletal muscles and possibly heart. In the context of the invention, when the gene therapy product is a viral vector comprising a polynucleotide encoding a human FKRP, the therapeutic dose is defined as the quantity of viral particles (vg for viral genomes) containing the FKRP sequence, administered per kilogram (kg) of the subject.

Available routes of administration are topical (local), enteral (system-wide effect, but delivered through the gastrointestinal (GI) tract), or parenteral (systemic action, but delivered by routes other than the GI tract). The preferred route of administration of the compositions disclosed herein is parenteral which includes intramuscular administration (i.e. into the muscle) and systemic administration (i.e. into the circulating system). In this context, the term “injection” (or “perfusion” or “infusion”) encompasses intravascular, in particular intravenous (IV), intramuscular (IM), intraocular, intrathecal or intracerebral administration. Injections are usually performed using syringes or catheters.

In one embodiment, systemic delivery of the composition comprises administering the composition near a local treatment site, i.e. in a vein or artery nearby a weakened muscle. In certain embodiments, the invention comprises the local delivery of the composition, which produces systemic effects. This route of administration, usually called “regional (loco-regional) infusion”, “administration by isolated limb perfusion” or “high-pressure transvenous limb perfusion” has been successfully used as a gene delivery method in muscular dystrophy.

According to one aspect, the composition is administered to an isolated limb (loco-regional) by infusion or perfusion. In other words, the invention comprises the regional delivery of the composition in a leg and/or arm by an intravascular route of administration, i.e. a vein (transveneous) or an artery, under pressure. This is usually achieved by using a tourniquet to temporarily arrest blood circulation while allowing a regional diffusion of the infused product, as e.g. disclosed by Toromanoff et al. (2008).

In one embodiment, the composition is injected in a limb of the subject. When the subject is a human, the limb can be the arm or the leg. According to one embodiment, the composition is administered in the lower part of the body of the subject, e.g. below the knee, or in the upper part of the body of the subject, e.g., below the elbow.

A preferred method of administration according to the invention is systemic administration. Systemic injection opens the way to an injection of the whole body, in order to reach the entire muscles of the body of the subject including the heart and the diaphragm and then a real treatment of these systemic and still incurable diseases. In certain embodiments, systemic delivery comprises delivery of the composition to the subject such that composition is accessible throughout the body of the subject.

According to a preferred embodiment, systemic administration occurs via injection of the composition in a blood vessel, i.e. intravascular (intravenous or intra-arterial) administration. According to one embodiment, the composition is administered by intravenous injection, through a peripheral vein.

The systemic administration is typically performed in the following conditions:
a flow rate of between 1 to 10 mL/min, advantageously between 1 to 5 mL/min, e.g. 3 mL/min;
the total injected volume can vary between 1 and 20 mL, preferably 5 mL of vector preparation per kg of the subject. The injected volume should not represent more than 10% of total blood volume, preferably around 6%.

When systemically delivered, the composition is preferably administered with a dose less than or equal to 10¹⁵ vg/kg or even 10¹⁴ vg/kg, advantageously superior or equal to 10¹⁰, 10¹¹, or even 10¹² vg/kg. Specifically, the dose can be between 5·10¹² vg/kg and 10¹⁴ vg/kg, e.g. 1, 2, 3, 4, 5, 6, 7, 8 or 9·10¹³ vg/kg. A lower dose of e.g. 1, 2, 3, 4, 5, 6, 7, 8 or 9·10¹² vg/kg can also be contemplated in order to avoid potential toxicity and/or immune reactions. As known by the skilled person, a dose as low as possible giving a satisfying result in term of efficiency is preferred.

In a specific embodiment, the treatment comprises a single administration of the composition.

“Dystroglycanopathy” means a disease or pathology linked to an aberrant glycosylation of α-dystroglycan (αDG). This defect can be due to a FKRP defect. According to a specific embodiment, the pathology is selected in the group consisting of: Limb-Girdle Muscular Dystrophy type 2I or type R9 (LGMD2I or LGMD2 R9), Congenital Muscular Dystrophy type 1C (MDC1C), Walker-Warburg Syndrome (WWS) and Muscle-Eye-Brain disease (MEB), advantageously LGMD2I.

Subjects that could benefit from the compositions of the invention include all patients diagnosed with such a disease or at risk of developing such a disease. A subject to be treated can then be selected based on the identification of mutations or deletions in the FKRP gene by any method known to the one skilled in the art, including for example sequencing of the FKRP gene, and/or through the evaluation of the FKRP level of expression or activity by any method known to the one skilled in the art. Therefore, said subjects include both subjects already exhibiting symptoms of such a disease and subjects at risk of developing said disease. In one embodiment, said subjects include subjects already exhibiting symptoms of such a disease. In another embodiment, said subjects are ambulatory patients and early non-ambulant patients.

Such compositions are notably intended for gene therapy, particularly for the treatment of Limb-Girdle Muscular Dystrophy type 2I (LGMD2I), Congenital Muscular Dystrophy type 1C (MDC1C), Walker-Warburg Syndrome (WWS) and Muscle-Eye-Brain disease (MEB), advantageously LGMD2I.

According to one embodiment, the present invention concerns a method of treatment of a dystroglycanopathy comprising administering to a subject the gene therapy product (expression system, vector or cell), as disclosed above.

Advantageously, the dystroglycanopathy is a pathology linked to an aberrant glycosylation of α-dystroglycan (αDG) and/or a FKRP deficiency. More advantageously, the pathology is Limb-Girdle Muscular Dystrophy type 2I (LGMD2I), Congenital Muscular Dystrophy type 1C (MDC1C), Walker-Warburg Syndrome (WWS) or Muscle-Eye-Brain disease (MEB).

In an additional aspect, the invention provides a method of increasing glycosylation of α-dystroglycan (αDG) in a cell comprising delivering to said cell the expression system or the vector of the invention, wherein the FKRP polynucleotide is expressed in said cell, thereby producing FKRP and increasing glycosylation of αDG.

Advantageously, the expression system is administered systemically in the body, particularly in an animal, advantageously in mammals and more preferably in humans.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

The disclosures of each and every patent, patent application, and publication cited herein are hereby incorporated herein by reference in their entirety.

Without further description, it is believed that one of ordinary skill in the art can, using the preceding description and the following illustrative examples, make and utilize the compounds of the present invention and practice the claimed methods.

### EXPERIMENTAL EXAMPLES

The invention is further described in detail by reference to the following experimental examples and the attached figures. These examples are provided for purposes of illustration only, and are not intended to be limiting.

In the application, the invention is illustrated in relation to an AAV9 vector comprising a sequence encoding FKRP placed under the control of the desmin promoter and one or two miR208a target sequence(s).

### FIGURES

FIG. 1: Diagram of the vector constructs:

A/ FKRP expression cassette, devoid of target sequences for miRNA-208a (AAV-FKRP);

B/ FKRP expression cassette containing 1 (AAV-FKRP-single) or 2 (AAV-FKRP-tandem) target sequences for miRNA-208a (arrow) at the 3′ end of the FKRP gene.

FIG. 2: Cross section of the heart of a rat intravenously administered with AAV-FKRP vector: Histological analysis of the heart muscle, at day 15 after injection of AAV-FKRP at 3 doses as indicated (1^{e}12 vg/kg; 5^{e}12 vg/kg; 7.5^{e}13 vg/kg) and HES staining (top, scale=50 μm) or Sirius red staining (bottom).

FIG. 3: Cross section of the heart of a mouse intravenously administered with AAV-FKRP vector: Histological analysis of the heart muscle, six weeks after injection of AAV-FKRP at dose 1^{e}14 vg/kg and HPS staining (top, scale=200 μm) or Sirius red staining (bottom).

FIG. 4: Body mass curve of rats injected with either PBS (buffer), AAV-FKRP, AV-FKRP-single or AAV-FKRP-tandem.

FIG. 5: Vector copy number (VCN) per nucleus of AAV-FKRP, AAV-FKRP-single and AAV-FKRP-tandem in the TA (tibialis anterior) muscle of rats 2 weeks after injection.

FIG. 6: Evaluation of FKRP mRNA (A) or protein (B) in the heart of rats 2 weeks after injection of PBS (buffer), AAV-FKRP, AV-FKRP-single or AAV-FKRP-tandem. The asterisk (*) indicates a statistic difference.

FIG. 7: Histological analysis of the heart muscle of rats at day 15 after injection of AAV-FKRP, AAV-FKRP-single or AAV-FKRP-tandem (as indicated) at dose 7.5^{e}13 vg/kg and HES staining (top, scale=50 μm) or Sirius red staining (bottom).

FIG. 8: Evaluation of FKRP mRNA (A) or protein (B) in the TA muscle of rats 2 weeks after injection of PBS (buffer), AAV-FKRP, AV-FKRP-single or AAV-FKRP-tandem.

FIG. 9: Body mass curve of rats injected with either AV-FKRP-single or AAV-FKRP-tandem.

FIG. 10: Histological analysis of the heart muscle of rats 11 weeks after injection of AAV-FKRP-single and AAV-FKRP-tandem at dose 7.5^{e}13 vg/kg and HES staining (top, scale=50 μm) or Sirius red staining (bottom).

### MATERIALS AND METHODS

1) Generation of Recombinant AAV Vectors:

The cassette contained in vector AAV-FKRP (SEQ ID NO: 1; see FIG. 1A) corresponds to nucleotides 496 to 4550 of the sequence SEQ ID NO: 11 as disclosed in WO2019/008157. Target sequences (1 or 2 sequences, respectively) of the miRNA-208a of 22 pb (SEQ ID NO: 2), each separated by DNA spacers, have been added in the 3′UTR region of the FKRP cDNA. The corresponding cassettes (FIG. 1B) have sequence SEQ ID NO: 3 and SEQ ID NO: 4, respectively, giving rise to vector AAV-FKRP-single and AAV-FKRP-tandem, respectively.

In detail, the expression cassette of SEQ ID NO: 1 contains:
5′ITR sequences corresponding to nucleotides 1 to 145 of SEQ ID NO: 1; followed by
the human desmin promoter (SEQ ID NO: 6) corresponding to nucleotides 146 to 1206 of SEQ ID NO: 1; followed by
the HBB2 intron corresponding to nucleotides 1207 to 1652 of SEQ ID NO: 1; followed by consensus Kozak sequence (GCCACC) inserted just before
the polynucleotide encoding the human FKRP (SEQ ID NO: 5) corresponding to nucleotides 1659 to 3146 of SEQ ID NO: 1; followed by
the HBB2 polyA sequence corresponding to nucleotides 3147 to 3912 of SEQ ID NO: 1; followed by
3′ITR sequences corresponding to nucleotides 3913 to 4057 of SEQ ID NO: 1.

In detail, the expression cassette of SEQ ID NO: 3 contains:
5′ITR sequences corresponding to nucleotides 1 to 145 of SEQ ID NO: 3; followed by
the human desmin promoter (SEQ ID NO: 6) corresponding to nucleotides 146 to 1206 of SEQ ID NO: 3; followed by
the HBB2 intron corresponding to nucleotides 1207 to 1652 of SEQ ID NO: 3; followed by consensus Kozak sequence (GCCACC) inserted just before
the polynucleotide encoding the human FKRP (SEQ ID NO: 5) corresponding to nucleotides 1659 to 3146 of SEQ ID NO: 3; followed by
a target sequence of miR208a (SEQ ID NO: 2) corresponding to nucleotides 3153 to 3174 of SEQ ID NO: 3; followed by
the HBB2 polyA sequence corresponding to nucleotides 3181 to 3946 of SEQ ID NO: 3; followed by
3′ITR sequences corresponding to nucleotides 3947 to 4091 of SEQ ID NO: 3.

In detail, the expression cassette of SEQ ID NO: 4 contains:
5′ITR sequences corresponding to nucleotides 1 to 145 of SEQ ID NO: 4; followed by
the human desmin promoter (SEQ ID NO: 6) corresponding to nucleotides 146 to 1206 of SEQ ID NO: 4; followed by
the HBB2 intron corresponding to nucleotides 1207 to 1652 of SEQ ID NO: 4; followed by consensus Kozak sequence (GCCACC) inserted just before
the polynucleotide encoding the human FKRP (SEQ ID NO: 5) corresponding to nucleotides 1659 to 3146 of SEQ ID NO: 4; followed by
two target sequence of miR208a (SEQ ID NO: 2) in tandem corresponding to nucleotides 3153 to 3174 and nucleotides 3181 to 3202 of SEQ ID NO: 4; followed by
the HBB2 polyA sequence corresponding to nucleotides 3209 to 3974 of SEQ ID NO: 4; followed by
3′ITR sequences corresponding to nucleotides 3975 to 4119 of SEQ ID NO: 4.

Adenovirus free rAAV2/9 viral preparations were generated by packaging AAV2-ITR recombinant genomes in AAV9 capsids, using a three plasmids transfection protocol as previously described (Bartoli et al., 2006). Briefly, HEK293 cells were cotransfected with pAAV-hDesmin-hFKRP, a RepCap plasmid (pAAV2.9, Dr J. Wilson, UPenn) and an adenoviral helper plasmid (pXX6; Apparailly et al., 2005) at a ratio of 1:1:2. Crude viral lysate was harvested at 60 hr post-transfection and lysed by freeze-and-thaw cycles. The viral lysate was purified through two rounds of CsCl ultracentrifugation followed by dialysis. Viral genomes were quantified by a TaqMan real-time PCR assay using primers and probes specific of the FKRP coding sequence contained in the AAV vector genome. The primer pairs and TaqMan probes used for amplification were:

FKRPopt Forward:
(SEQ ID NO: 9)
GCCCTTCTACCCCAGGAATG
FKRPopt Reverse:
(SEQ ID NO: 10)
AAACTTCAGCTCCAGGAACCTC;
and
FKRPopt Probe:
(SEQ ID NO: 11)
TGCCCTTTGCTGGCTTTGTGGCCCAGGC.

The vector titres are expressed in terms of viral genomes per ml (vg/ml).

2) In Vivo Experiments:

The rats and mice were treated according to the French and European legislation regarding animal testing. In this study, Sprague-Dawley male rats 10-12 weeks old and male FKRP-deficient mice (Gicquel et al., P094, Conférence European Society Of Gene & Cell Therapy 2017, doi: 10.1089/hum.2017.29055.abstracts) 4 weeks old were used. Recombinant vectors, as per the indicated doses, were injected into the tail vein of the rats and mice as indicated. An equivalent volume of saline buffer (PBS) was administered as a control. The clinical status and animal weight were monitored on a regular basis. The animals were sacrificed at the indicated times (2 weeks or 11 weeks for the rats; 6 weeks for the mice).

3) Western Blot:

Heart and muscle tissues were mechanically homogenized in RIPA lysis buffer (Thermo Fisher Scientific, Waltham, Mass., USA), complemented with Complete protease inhibitor cocktail EDTA-free (Roche, Bale, Switzerland). Nucleic acids contained in the samples were degraded by incubation 15 minutes at 37° C. with benzonase (Sigma, St. Louis, Mo., USA).

Proteins were separated using precast polyacrylamide gel (4-15%, BioRad, Hercules, Calif., USA) and then transferred to nitrocellulose membrane.

Rabbit polyclonal antibody against FKRP has been previously described (Gicquel et al., 2017). Nitrocellulose membranes were probed with antibodies against FKRP (1:100) and GAPDH (Santa Cruz Biotechnologies, Dallas, Tex., USA, 1:5000) for normalization, for 2 hours at room temperature.

Finally, membranes were incubated with IRDye® for detection by the Odyssey infrared-scanner (LI-COR Biosciences, Lincoln, Nebr., USA).

4) PCR:

Vector copy number (VCN) were quantified in TA muscle by quantitative RT-PCR on HBB2 polyA sequence contained in the vector genome, and normalized using the titin gene (TTN).

HBB2pA Forward:
(SEQ ID NO: 12)
CTTGACTCCACTCAGTTCTCTTGCT;
HBB2pA Reverse:
(SEQ ID NO: 13)
CCAGGCGAGGAGAAACCA;
and
HBB2pA Probe:
(SEQ ID NO: 14)
CTCGCCGTAAAACATGGAAGGAACACTTC.
TTN Forward:
(SEQ ID NO: 15)
GTCCCCTGCGTATCTGCTATG;
TTN Reverse:
(SEQ ID NO: 16)
CGCTCGTTTTCAATACTACCTCTCT;
and
TTN Probe:
(SEQ ID NO: 17)
TCCGCAGCTCTAGTGGAAGAACCACC.

FKRP mRNA was extracted from TA muscle and from heart using the TriZOL method, then quantified by quantitative RT-PCR using oligonucleotides and probe designed on the codon-optimized FKRP sequence, and normalized by the expression of P0 gene.

P0 Forward:
(SEQ ID NO: 18)
CTCCAAGCAGATGCAGCAGA;
P0 Reverse:
(SEQ ID NO: 19)
ATAGCCTTGCGCATCATGGT;
and
P0 Probe:
(SEQ ID NO: 20)
CCGTGGTGCTGATGGGCAAGAA.

FKRPopt Forward (SEQ ID NO: 9), FKRPopt Reverse (SEQ ID NO: 10) and FKRPopt Probe (SEQ ID NO: 11) are as disclosed above.

5) Histology:

Cross cryosections (8 μm thickness) of the cardiac muscle were stained with Hematoxyline-Eosin-Saffran (HES), sinus red or Hematoxyline-Phloxin-Saffron (HFS) using standard protocols.

The sections were mounted with the PERTEX medium (Leica). The digital images were captured using Axio Scan Z1 slide scanner (Zeiss).

Results:

1/ Fkrp Gene Transfer Induces Cardiac Toxicity

1-1/ In Rats

Systemic administration of AAV-FKRP (FIG. 1A; harboring SEQ ID NO: 1) was performed in 5 male rats (Sprague-Dawley), 10-12 weeks old, at 3 different doses: 1^{e}12, 5^{e}12 and 7.5^{e}13 vg/kg. Two weeks after injection, the rats were euthanized and sampled. Slices of hearts were stained both with Hematoxyline-Eosin-Saffran (HES) and with Sirius red.

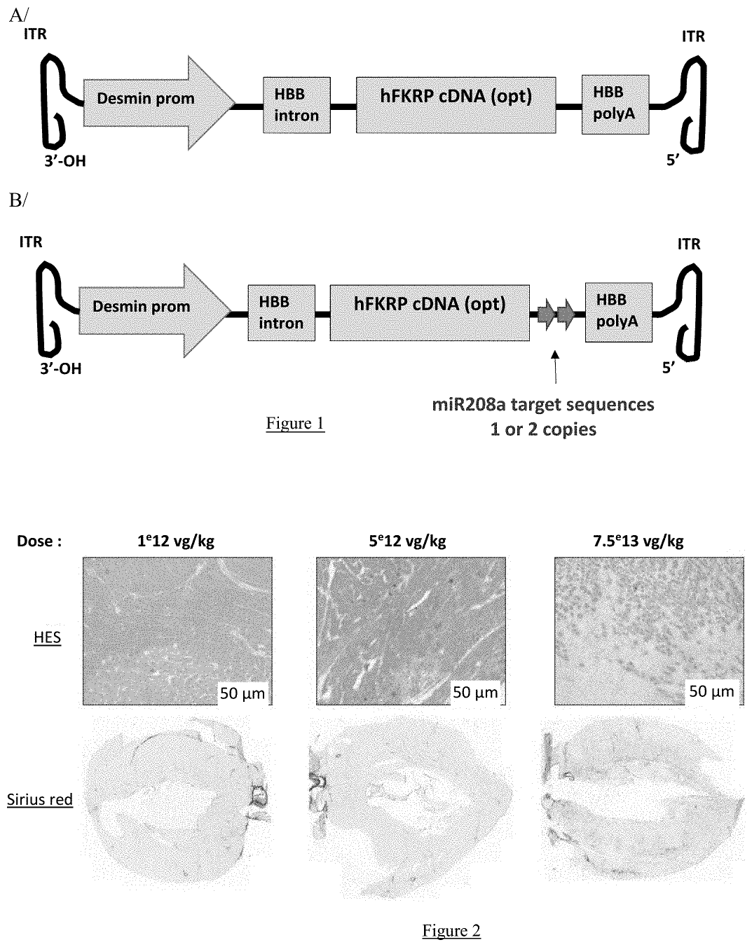

Histology of rat hearts after AAV-FKRP administration show cardiac damages: as shown in FIG. 2, inflammation and fibrosis are clearly observed in rats at day 15 after injection at dose 7.5^{e}13 vg/kg. Moreover, in these conditions, one rat died.

1-2/ In Mice

Since mouse is the only mammal species in which a FKRP-deficient animal model has been developed and therefore the only species in which the therapeutic effect of expression systems can be explored, the potential cardiac toxicity of the AAV-FKRP vector was also investigated in this model.

Systemic administration of AAV-FKRP was performed in 6 male FKRP-deficient mice, 4 weeks old, at 4 doses: 5^{e}12, 1.5^{e}13, 4.5^{e}13 and 1^{e}14 vg/kg. Six weeks after injection, the mice were euthanized and sampled. Slices of hearts were stained both with Hematoxyline-Phloxin-Saffran (HPS) and with Sirius red.

All mice survived to the study, even for the highest dose (1^{e}14 vg/kg). On the contrary (see below), 1 rat died 2 weeks after administration at dose from 7.5^{e}13 vg/kg. This reveals that mice are less affected than rats by AAV-FKRP systemic administration.

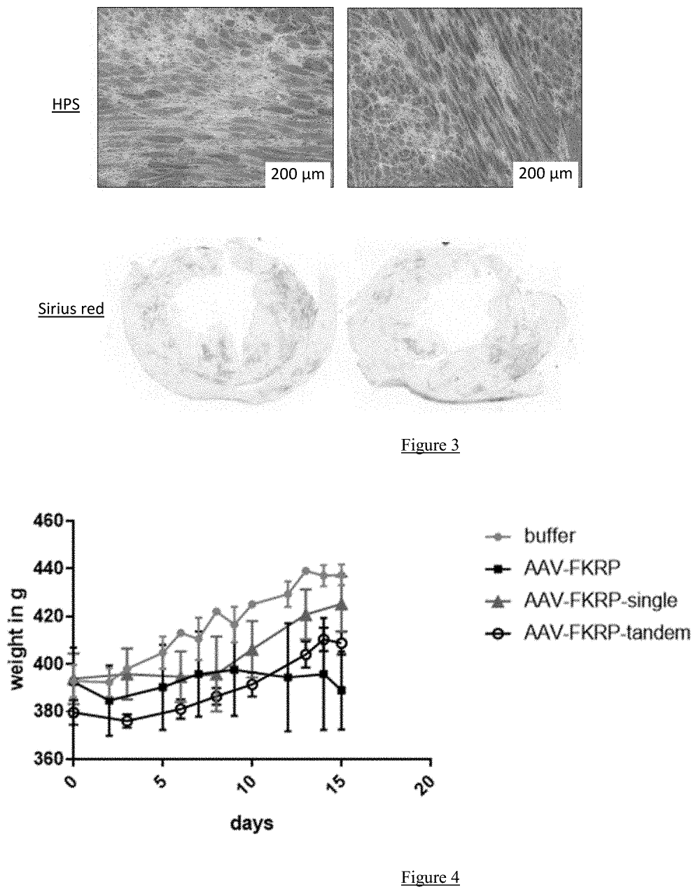

However, histology of mice hearts after AAV-FKRP administration reveals cardiac damages: as shown in FIG. 3, inflammation and fibrosis are observed in mice 6 weeks after injection at dose 1^{e}14 vg/kg.

As a whole, the presented data reveal a cardiac toxic effect of AAV-FKRP, which is confirmed in 2 species (rat and mouse) and which was fully unexpected.

2/ Decreasing Fkrp Transgene Expression in the Heart Alleviates Cardiac Toxicity without Affecting Muscular Expression

As a proof of concept to prevent FKRP cardiac toxicity, one or two copies of the target sequence of a cardiac specific micro-RNA, i.e. miR-208a, were introduced in the AAV-FKRP vector. The so obtained vectors (FIG. 1B) are named AAV-FKRP-single (containing one target sequence of miR-208a and harboring SEQ ID NO: 3) and AAV-FKRP-tandem (containing two target sequences of miR-208a in the same direction and harboring SEQ ID NO: 4).

2-1/ Short-Term (2 Weeks) Test in Rats

Based on the previous data, the rat model was chosen for further experiments because this animal model reveals heart toxicity in a rapid and clear manner, especially at dose 7.5′13 vg/kg.

Systemic administration of AAV-FKRP containing 0, 1 or 2 copies of miR-208a target (SEQ ID NO: 2) was performed in 5 male rats (Sprague-Dawley), 10-12 weeks old, at the dose of 7.5′13 vg/kg. Two weeks after injection, the rats were euthanized and sampled.

a) Survival and Weight Follow Up:

The survival data are shown in the Table below:

| Injected (i.v.) | Survival |
|---|---|
| Buffer | 5/5 |
| AAV-FKRP | 4/5 |
| AAV-FKRP-single | 5/5 |
| AAV-FKRP-tandem | 5/5 |

The data reveal that the only death occurred in the cohort administered with AAV-FKRP, probably because of the cardiac toxicity of this construct.

Moreover, FIG. 4 shows that rats injected with AAV-FKRP do not gain weight with time whereas rats with AAV-FKRP-single or with AAV-FKRP-tandem do.

As a conclusion and after 2 weeks, it appears that the rats administered with AAV-FKRP-single or AAV-FKRP-tandem are fitter than those administered with AAV-FKRP.

b) Vector Copy Number Quantification in TA Muscle:

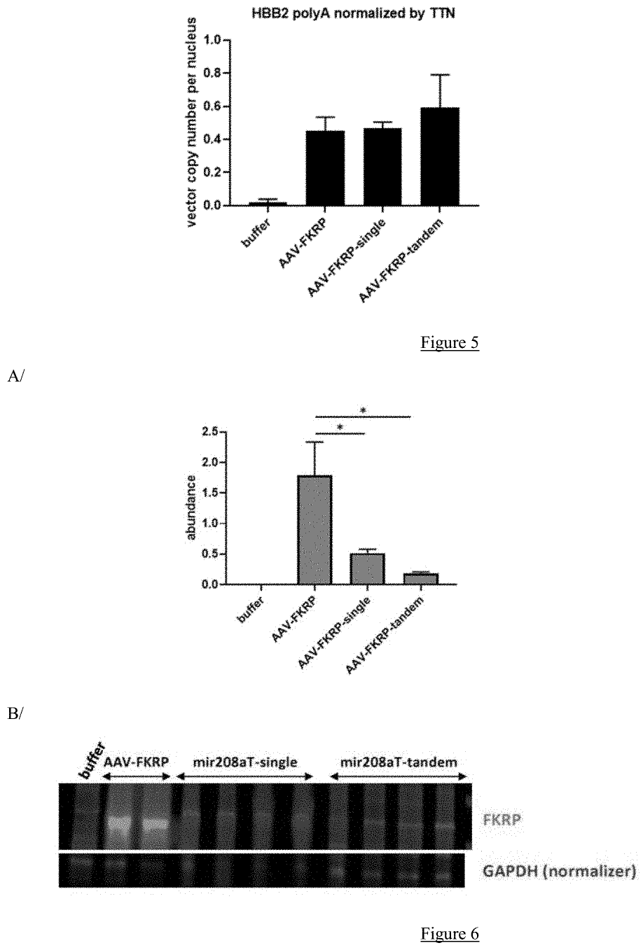

The data shown in FIG. 5, based on the quantification of the HBB2 polyA sequence contained in each vector genome further normalized using the titin gene (TTN), reveal a similar level of infection of the skeletal muscle tissue, i.e. the TA muscle, with the 3 vectors.

Importantly, this confirms that the introduction of the miR208a target sequence(s) does not have any negative impact on the efficiency of the vector transfer in muscles, wherein said protein should be produced at a therapeutic level to cure the muscular abnormalities associated with a deficiency of FKRP.

c) FKRP Expression in the Heart after Gene Transfer

As shown in FIG. 6, at the mRNA level (A) as well as at the protein level (B), an important decrease of FKRP transgene expression is observed with the constructs AAV-FKRP-single and AAV-FKRP-tandem compared to AAV-FKRP.

It is to be noted that one miR208a target sequence is sufficient to observe such a decrease.

d) Heart Damages after Gene Transfer

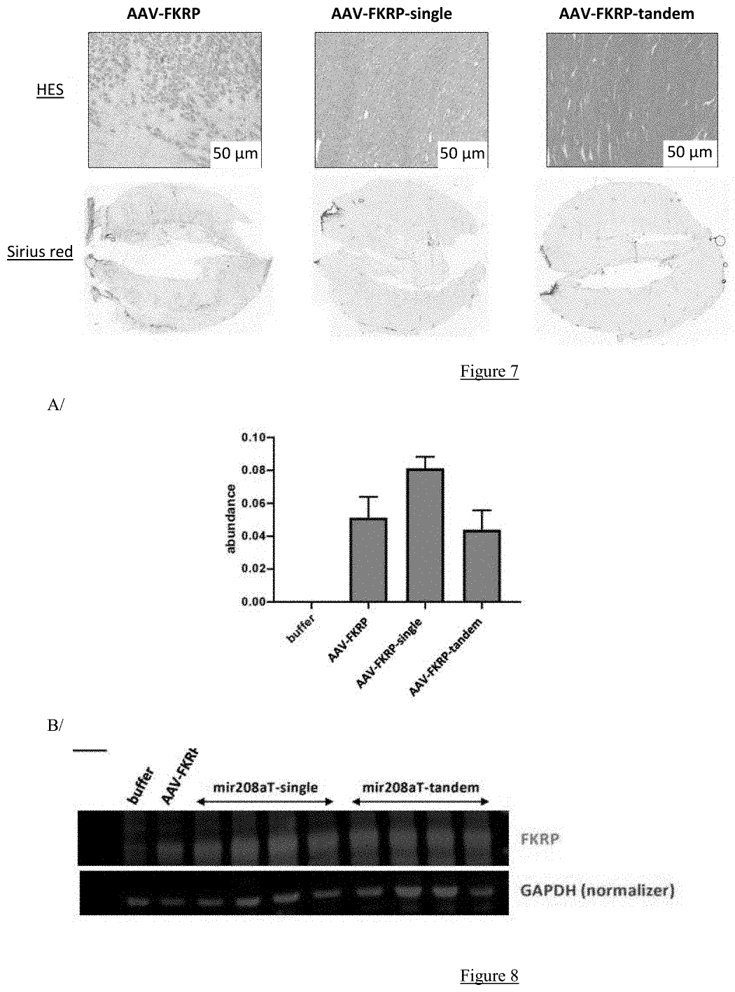

The data shown in FIG. 7 reveal a huge decrease of heart damages with the constructs AAV-FKRP-single and AAV-FKRP-tandem in comparison to AAV-FKRP. In other words, the toxic effect disappears when FKRP transgene expression is reduced in the heart, i.e. using regulation by adequate micro-RNA.

e) FKRP Expression in the Skeletal Muscle after Gene Transfer

As shown in FIG. 8 in relation to the TA muscle, at the mRNA level (A) as well as at the protein level (B), no decrease of FKRP transgene expression is observed with the constructs AAV-FKRP-single and AAV-FKRP-tandem compared to AAV-FKRP.

This confirms that the use of miR208a allows to specifically detarget the heart. It is of high importance that the introduction of the miR208a target sequence(s) does not have any negative impact on the efficiency of the FKRP expression in skeletal muscles, wherein said protein should be produced at a therapeutic level to cure the muscular abnormalities associated with a deficiency thereof

2-2/ Long-Term (11 Weeks) Test in Rats

The same experiments as reported above have been performed on rats but 11 weeks after injection.

a) Survival and Weight Follow Up:

As a reminder, at sacrifice 2 weeks after administration with AAV-FKRP, 1 rat was died whereas all had severe cardiac damages. On the contrary, all the rats injected with AAV-FKRP-single or with AAV-FKRP-tandem survived even 11 weeks after administration.

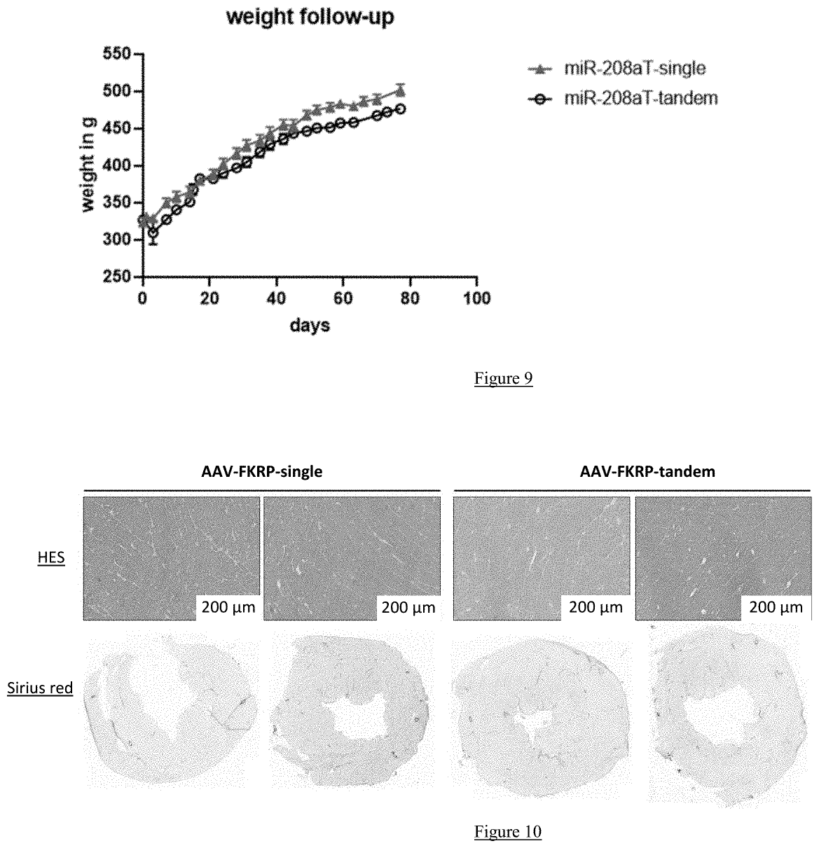

Moreover, FIG. 9 shows that rats injected with AAV-FKRP-single or with AAV-FKRP-tandem do gain weight with time.

As a conclusion and after 11 weeks, it appears that all the rats administered with AAV-FKRP-single or AAV-FKRP-tandem are in good shape.

b) Heart Damages after Gene Transfer:

Moreover, FIG. 10 confirms that even after 11 weeks, no heart damage is observed.

In conclusion, vectors AAV-FKRP-single and AAV-FKRP-tandem do not display any cardiac toxicity.

### REFERENCES

Apparailly, F., Khoury, M., Vervoordeldonk, M. J., Adriaansen, J., Gicquel, E., Perez, N., Riviere, C., Louis-Plence, P., Noel, D., Danos, O. et al. (2005) Adeno-associated virus pseudotype 5 vector improves gene transfer in arthritic joints. Hum. Gene Ther., 16, 426-434.
Bartoli, M., Poupiot, J., Goyenvalle, A., Perez, N., Garcia, L., Danos, O. and Richard, I. (2006) Noninvasive monitoring of therapeutic gene transfer in animal models of muscular dystrophies. Gene Ther., 13, 20-28.
Beltran-Valero de Bernabe, D., Voit, T., Longman, C., Steinbrecher, A., Straub, V., Yuva, Y., Herrmann, R., Sperner, J., Korenke, C., Diesen, C. et al. (2004) Mutations in the FKRP gene can cause muscle-eye-brain disease and Walker-Warburg syndrome. J. Med. Genet., 41, e61.
Breton, C. and Imberty, A. (1999) Structure/function studies of glycosyltransferases. Curr. Opin. Struct. Biol., 9, 563-571.
Brockington, M., Blake, D. J., Prandini, P., Brown, S. C., Torelli, S., Benson, M. A., Ponting, C. P., Estournet, B., Romero, N. B., Mercuri, E. et al. (2001) Mutations in the fukutin-related protein gene (FKRP) cause a form of congenital muscular dystrophy with secondary laminin alpha2 deficiency and abnormal glycosylation of alpha-dystroglycan. Am. J. Hum. Genet., 69, 1198-1209.
Gicquel et al. (2017) Hum Mol Genet, doi: 10.1093/hmg/ddx066.
Kanagawa, M., Kobayashi, K., Tajiri, M., Manya, H., Kuga, A., Yamaguchi, Y., Akasaka-Manya, K., Furukawa, J. I., Mizuno, M., Kawakami, H. et al. (2016) Identification of a Post-translational Modification with Ribitol-Phosphate and Its Defect in Muscular Dystrophy. Cell reports, in press.
Mercuri, E., Brockington, M., Straub, V., Quijano-Roy, S., Yuva, Y., Herrmann, R., Brown, S. C., Torelli, S., Dubowitz, V., Blake, D. J. et al. (2003) Phenotypic spectrum associated with mutations in the fukutin-related protein gene. Ann. Neurol., 53, 537-542.
Muller, T., Krasnianski, M., Witthaut, R., Deschauer, M. and Zierz, S. (2005) Dilated cardiomyopathy may be an early sign of the C826A Fukutin-related protein mutation. Neuromuscul. Disord., 15, 372-376.
Petri et al. (2015), International Journal of Cardiology, 182 (2015) 403-411.
Rosales et al. (2011), Journal of Cardiovascular Magnetic Resonance, 13:39.
Sveen, M. L., Schwartz, M. and Vissing, J. (2006) High prevalence and phenotype-genotype correlations of limb girdle muscular dystrophy type 2I in Denmark. Ann. Neurol., 59, 808-815.
Toromanoff et al. (2008), Molecular Therapy 16(7):1291-99.
Wahbi, K., Meune, C., Hamouda el, H., Stojkovic, T., Laforet, P., Becane, H. M., Eymard, B. and Duboc, D. (2008) Cardiac assessment of limb-girdle muscular dystrophy 21 patients: an echography, Holter ECG and magnetic resonance imaging study. Neuromuscul. Disord., 18, 650-655.

1-15. (canceled)

16. An expression system for systemic administration comprising a sequence encoding a Fukutin-related protein (FKRP), and:
a promoter sequence allowing the expression at a therapeutically acceptable level of FKRP in the skeletal muscles and a target sequence of an miRNA expressed in the heart; or
a promoter sequence allowing the expression at a therapeutically acceptable level of FKRP in the skeletal muscles and presenting a promoter activity at a toxically acceptable level in the heart.

17. The expression system according to claim 16, wherein the FKRP protein has the sequence SEQ ID NO: 5.

18. The expression system according to claim 16, wherein the sequence encoding the FKRP protein comprises nucleotides 1659 to 3146 of SEQ ID NO: 1, SEQ ID NO: 3, or SEQ ID NO: 4.

19. The expression system according to claim 16, wherein the expression system comprises at least one target sequence of miR208a.

20. The expression system according to claim 19, wherein the target sequence of miR208a comprises the sequence SEQ ID NO: 2.

21. The expression system according to claim 16, wherein the expression system comprises a desmin promoter.

22. The expression system according to claim 21, wherein the desmin promoter comprises a sequence as set forth in SEQ ID NO: 6.

23. The expression system according to claim 21, wherein the expression system comprises nucleotides 146 to 3946 of SEQ ID NO: 3, or nucleotides 146 to 3974 of SEQ ID NO: 4.

24. The expression system according to claim 16, wherein the expression system comprises a promoter sequence of a calpain 3 gene or a promoter sequence of miR206.

25. The expression system according to claim 24, wherein the promoter sequence of the calpain 3 gene comprises a sequence as set forth in SEQ ID NO: 7.

26. The expression system according to claim 24, wherein the promoter sequence of the miR206 comprises a sequence as set forth in SEQ ID NO: 8.

27. The expression system according to claim 16, wherein the expression system comprises a vector having a tropism higher for skeletal muscles than for heart muscles.

28. The expression system according to claim 16, wherein the expression system comprises a viral vector.

29. The expression system according to claim 28, wherein the viral vector is an adeno-associated viral vector (AAV).

30. The expression system according to claim 29, wherein the AAV is of serotype 8 or serotype 9.

31. The expression system according to claim 29, wherein the expression system comprises an AAV2/8 or an AAV2/9 vector.

32. A pharmaceutical composition comprising an expression system according to claim 16.

33. A method of treating a pathology linked to a FKRP deficiency or induced by a defect in α-dystroglycan (α-DC) glycosylation in a subject, the method comprising:
administering a composition according to claim 32 to the subject,
wherein the pathology is selected from the group consisting of: Limb-Girdle Muscular Dystrophy type 2I (LGMD2I), Congenital Muscular Dystrophy type 1C (MDC1C), Walker-Warburg Syndrome (WWS) and Muscle-Eye-Brain disease (MEB), advantageously LGMD2I.

34. The method according to claim 33, wherein the composition is administered systemically.

35. The method according to claim 33, wherein the composition is administered by intravenous injection.


SEQUENCE LISTING (21 sequences)

SEQ ID NO: 1 - DNA, 4057 bp, Artificial Sequence
  Synthetic Polynucleotide
  misc_feature: K7 AAV-FKRP
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttttggttgggataaggctggattattctgagtccaagctaggcccttttgctaatcttgttcatacctcttatcttcctcccacagctcctgggcaacgtgctggtctctgtgctggcccatcactttggcaaagaattcgccaccatgagactgaccaggtgccaggctgccctggctgctgccatcaccctgaacctgctggtgctgttctatgtgagctggctgcagcaccagcccaggaacagcagggccaggggccccaggagggcctctgctgctggccccagggtgacagtgctggtgagggagtttgaggcctttgacaatgctgtgcctgagctggtggacagcttcctgcagcaggaccctgcccagcctgtggtggtggctgctgataccctgccctacccccccctggccctgcccaggatccccaatgtgaggctggccctgctgcagcctgccctggacaggcctgctgctgccagcaggcctgagacctatgtggccacagagtttgtggccctggtgcctgatggggccagggctgaggcccctggcctgctggagaggatggtggaggccctgagggctggctctgccaggctggtggctgcccctgtggccacagccaaccctgccaggtgcctggccctgaatgtgagcctgagagagtggacagccaggtatggggctgcccctgctgcccccaggtgtgatgccctggatggagatgctgtggtgctgctgagggccagggacctgttcaacctgtctgcccccctggccaggcctgtggggaccagcctgtttctgcagacagccctgaggggctgggctgtgcagctgctggacctgacctttgctgctgccaggcagccccccctggctacagcccacgccaggtggaaggctgagagggagggcagggccaggagggctgccctgctgagggccctggggatcaggctggtgagctgggaggggggcaggctggagtggtttggctgcaacaaggagacaaccaggtgctttgggacagtggtgggggatacccctgcctacctgtatgaggagaggtggacccccccctgctgcctgagggccctgagggagacagccaggtatgtggtgggggtgctggaggctgctggggtgaggtactggctggaggggggcagcctgctgggggctgccaggcacggggacattatcccctgggactatgatgtggacctgggcatctacctggaggatgtgggcaactgtgagcagctgaggggggctgaggctggctctgtggtggatgagaggggctttgtgtgggagaaggctgtggagggggactttttcagggtgcagtactctgagagcaaccacctgcacgtggacctgtggcccttctaccccaggaatggggtgatgaccaaggacacctggctggaccacaggcaggatgtggagttccctgagcacttcctgcagcccctggtgcccctgccctttgctggctttgtggcccaggcccccaacaactacaggaggttcctggagctgaagtttggccctggggtgattgagaacccccagtaccccaaccctgccctgctgagcctgacaggctctggctgaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - DNA, 22 bp, Artificial Sequence
  Synthetic Polynucleotide
  misc_feature: miR208a target sequence
acaagctttttgctcgtcttat

SEQ ID NO: 3 - DNA, 4091 bp, Artificial Sequence
  Synthetic Polynucleotide
  misc_feature: K7 AAV-FKRP-single
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttttggttgggataaggctggattattctgagtccaagctaggcccttttgctaatcttgttcatacctcttatcttcctcccacagctcctgggcaacgtgctggtctctgtgctggcccatcactttggcaaagaattcgccaccatgagactgaccaggtgccaggctgccctggctgctgccatcaccctgaacctgctggtgctgttctatgtgagctggctgcagcaccagcccaggaacagcagggccaggggccccaggagggcctctgctgctggccccagggtgacagtgctggtgagggagtttgaggcctttgacaatgctgtgcctgagctggtggacagcttcctgcagcaggaccctgcccagcctgtggtggtggctgctgataccctgccctacccccccctggccctgcccaggatccccaatgtgaggctggccctgctgcagcctgccctggacaggcctgctgctgccagcaggcctgagacctatgtggccacagagtttgtggccctggtgcctgatggggccagggctgaggcccctggcctgctggagaggatggtggaggccctgagggctggctctgccaggctggtggctgcccctgtggccacagccaaccctgccaggtgcctggccctgaatgtgagcctgagagagtggacagccaggtatggggctgcccctgctgcccccaggtgtgatgccctggatggagatgctgtggtgctgctgagggccagggacctgttcaacctgtctgcccccctggccaggcctgtggggaccagcctgtttctgcagacagccctgaggggctgggctgtgcagctgctggacctgacctttgctgctgccaggcagccccccctggctacagcccacgccaggtggaaggctgagagggagggcagggccaggagggctgccctgctgagggccctggggatcaggctggtgagctgggaggggggcaggctggagtggtttggctgcaacaaggagacaaccaggtgctttgggacagtggtgggggatacccctgcctacctgtatgaggagaggtggacccccccctgctgcctgagggccctgagggagacagccaggtatgtggtgggggtgctggaggctgctggggtgaggtactggctggaggggggcagcctgctgggggctgccaggcacggggacattatcccctgggactatgatgtggacctgggcatctacctggaggatgtgggcaactgtgagcagctgaggggggctgaggctggctctgtggtggatgagaggggctttgtgtgggagaaggctgtggagggggactttttcagggtgcagtactctgagagcaaccacctgcacgtggacctgtggcccttctaccccaggaatggggtgatgaccaaggacacctggctggaccacaggcaggatgtggagttccctgagcacttcctgcagcccctggtgcccctgccctttgctggctttgtggcccaggcccccaacaactacaggaggttcctggagctgaagtttggccctggggtgattgagaacccccagtaccccaaccctgccctgctgagcctgacaggctctggctgatctagaacaagctttttgctcgtcttatctcgagattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 4 - DNA, 4119 bp, Artificial Sequence
  Synthetic Polynucleotide
  misc_feature: K7 AAV-FKRP-tandem
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttttggttgggataaggctggattattctgagtccaagctaggcccttttgctaatcttgttcatacctcttatcttcctcccacagctcctgggcaacgtgctggtctctgtgctggcccatcactttggcaaagaattcgccaccatgagactgaccaggtgccaggctgccctggctgctgccatcaccctgaacctgctggtgctgttctatgtgagctggctgcagcaccagcccaggaacagcagggccaggggccccaggagggcctctgctgctggccccagggtgacagtgctggtgagggagtttgaggcctttgacaatgctgtgcctgagctggtggacagcttcctgcagcaggaccctgcccagcctgtggtggtggctgctgataccctgccctacccccccctggccctgcccaggatccccaatgtgaggctggccctgctgcagcctgccctggacaggcctgctgctgccagcaggcctgagacctatgtggccacagagtttgtggccctggtgcctgatggggccagggctgaggcccctggcctgctggagaggatggtggaggccctgagggctggctctgccaggctggtggctgcccctgtggccacagccaaccctgccaggtgcctggccctgaatgtgagcctgagagagtggacagccaggtatggggctgcccctgctgcccccaggtgtgatgccctggatggagatgctgtggtgctgctgagggccagggacctgttcaacctgtctgcccccctggccaggcctgtggggaccagcctgtttctgcagacagccctgaggggctgggctgtgcagctgctggacctgacctttgctgctgccaggcagccccccctggctacagcccacgccaggtggaaggctgagagggagggcagggccaggagggctgccctgctgagggccctggggatcaggctggtgagctgggaggggggcaggctggagtggtttggctgcaacaaggagacaaccaggtgctttgggacagtggtgggggatacccctgcctacctgtatgaggagaggtggacccccccctgctgcctgagggccctgagggagacagccaggtatgtggtgggggtgctggaggctgctggggtgaggtactggctggaggggggcagcctgctgggggctgccaggcacggggacattatcccctgggactatgatgtggacctgggcatctacctggaggatgtgggcaactgtgagcagctgaggggggctgaggctggctctgtggtggatgagaggggctttgtgtgggagaaggctgtggagggggactttttcagggtgcagtactctgagagcaaccacctgcacgtggacctgtggcccttctaccccaggaatggggtgatgaccaaggacacctggctggaccacaggcaggatgtggagttccctgagcacttcctgcagcccctggtgcccctgccctttgctggctttgtggcccaggcccccaacaactacaggaggttcctggagctgaagtttggccctggggtgattgagaacccccagtaccccaaccctgccctgctgagcctgacaggctctggctgatctagaacaagctttttgctcgtcttatcctaggacaagctttttgctcgtcttatctcgagattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 5 - protein, 495 aa, Homo sapiens
  FKRP
MRLTRCQAALAAAITLNLLVLFYVSWLQHQPRNSRARGPRRASAAGPRVTVLVREFEAFDNAVPELVDSFLQQDPAQPVVVAADTLPYPPLALPRIPNVRLALLQPALDRPAAASRPETYVATEFVALVPDGARAEAPGLLERMVEALRAGSARLVAAPVATANPARCLALNVSLREWTARYGAAPAAPRCDALDGDAVVLLRARDLFNLSAPLARPVGTSLFLQTALRGWAVQLLDLTFAAARQPPLATAHARWKAEREGRARRAALLRALGIRLVSWEGGRLEWFGCNKETTRCFGTVVGDTPAYLYEERWTPPCCLRALRETARYVVGVLEAAGVRYWLEGGSLLGAARHGDIIPWDYDVDLGIYLEDVGNCEQLRGAEAGSVVDERGFVWEKAVEGDFFRVQYSESNHLHVDLWPFYPRNGVMTKDTWLDHRQDVEFPEHFLQPLVPLPFAGFVAQAPNNYRRFLELKFGPGVIENPQYPNPALLSLTGSG

SEQ ID NO: 6 - DNA, 1061 bp, Artificial Sequence
  Synthetic Polynucleotide
  misc_feature: Desmin promoter
taccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgc

SEQ ID NO: 7 - DNA, 1654 bp, Artificial Sequence
  Synthetic Polynucleotide
  misc_feature: Calpain 3 promoter
cacatgcctccactctgccatacttgaaatgtgctcatctccttacagcccagggagcagctattgtgggtagaagacaaggtggaggccaggcaggcacttcccttccccagagccacttatgctctcatctaagagccctgaaaccaggtgtgacatcccaggagttgacagacagtctggttcagtatctaattccaacttctgtctcagatgcctaatgtggcatggctgaatgagtcaacatataacctgtacagtaagtcctcacttaacatcattgataggtgcttgtaaactgtgactttaacgaaaacataccgtgtgctgtagggacttaactcttgtttatatcagttagcctggtttcactatacagtacatcattttgcttaaagtcacagcttacgagaacctatcgatgatgttaagtgaggattttctctgctcaggtgcactttttttttttttttaagacggagtctctttctgtcacctgggctggagtgcagtggcgcgatctgggttcactacaacctctgcctcctgggttcaagcaattcttctgtctcagcctcccaagtagctgggattacaggcacccgccgccacacccggcttatttttgtatttttagtagagacagggtttcactattgttggccatgctggtctcgaactcctgacctcatgtgatccacccgcctcggcctcccaaagtgcagagattagagacgtgagccacatggcccagcaggaccactttttagcagattcagtcccagtgttcattttgtggatggggagagacaagaggtggcaaggtcaagtgtgcaggtagagacagggattttctcaaatgaggactctgctgagtagcattttccatgcagacatttccaatgagcgctgacccaagaacattctaaaaaagataccaaatctaacattgaataatgttctgatatcctaaaattttaggactaaaaatcatgttctctaaaattcacagaatatttttgtagaattcagtacctcccgttcaccctaactagcttttttgcaatattgttttccattcatttgatggccagtagttgggtggtctgtataactgcctactcaataacatgtcagcagttctcagcttctttccagtgttcaccttactcagatactcccttttcattttctggcaacaccagcacttcatggcaacagaaatgtccctagccaggttctctctctaccatgcagtctctcttgctctcatactcacagtgtttcttcacatctatttttagttttcctggctcaagcatcttcaggccactgaaacacaaccctcactctctttctctctccctctggcatgcatgctgctggtaggagacccccaagtcaacattgcttcagaaatcctttagcactcatttctcaggagaacttatggcttcagaatcacagctcggtttttaagatggacataacctgtacgaccttctgatgggctttcaactttgaactggatgtggacacttttctctcagatgacagaattactccaacttcccctttgcagttgcttcctttccttgaaggtagctgtatcttattttctttaaaaagctttttcttccaaagccacttgcca

SEQ ID NO: 8 - DNA, 805 bp, Artificial Sequence
  Synthetic Polynucleotide
  misc_feature: miR206 promoter
gggggccaactcttcctttggcatatgtttccccattttctggcagagaatcagataccacaaagttcaaaaccccatctccctccagccagggtggccatccagaccctgagtggctcaacagctgccaatgtccctcatccttctgaggctcaggcctcacagattgtggggcaggtgatgggctagggggagcagaagcccgacaaaaggatccttcccacagtgaacaatggtgcttggaatgctggatgggcagctgctgcccatcaacaagcacccaaaacagatagacgtacagtaggaagtacaggagggccggtgtgtttctaagcatgagtggctctctgcgtgaatgtggaaaatttctctgttggattctctcttctttttaattttcccttcactggatcccaaacattaaaaaagaatcacattcaaaatgcacaaaaacagcagcagtgaattaattagtagtaataacaaaggactggatagactgtagctgcacaagaataagccagggaaacgtggtgctgcttatctgtgaacaaacagtaggaaggatttggtcccaagcagcactgccattcctcacaacagatttatttcagcatgatttggtcgggcgggggggatttaggatgagttgagatcccagtgatcttctcgctaagagtttcctgcctgggcaaggaggaaagatgctacaagtggcccacttctgagatgcgggctgcttctggatgacactgcttcccgaggccacatgcttctttatatccccatatggattactttgcta

SEQ ID NO: 9 - DNA, 20 bp, Artificial Sequence
  Synthetic Polynucleotide
  misc_feature: FKRPopt Forward
gcccttctaccccaggaatg

SEQ ID NO: 10 - DNA, 22 bp, Artificial Sequence
  Synthetic Polynucleotide
  misc_feature: FKRPopt Reverse
aaacttcagctccaggaacctc

SEQ ID NO: 11 - DNA, 28 bp, Artificial Sequence
  Synthetic Polynucleotide
  misc_feature: FKRPopt Probe
tgccctttgctggctttgtggcccaggc

SEQ ID NO: 12 - DNA, 25 bp, Artificial Sequence
  Synthetic Polynucleotide
  misc_feature: HBB2pA Forward
cttgactccactcagttctcttgct

SEQ ID NO: 13 - DNA, 18 bp, Artificial Sequence
  Synthetic Polynucleotide
  misc_feature: HBB2pA Reverse
ccaggcgaggagaaacca

SEQ ID NO: 14 - DNA, 29 bp, Artificial Sequence
  Synthetic Polynucleotide
  misc_feature: HBB2pA Probe
ctcgccgtaaaacatggaaggaacacttc

SEQ ID NO: 15 - DNA, 21 bp, Artificial Sequence
  Synthetic Polynucleotide
  misc_feature: TTN Forward
gtcccctgcgtatctgctatg

SEQ ID NO: 16 - DNA, 25 bp, Artificial Sequence
  Synthetic Polynucleotide
  misc_feature: TTN Reverse
cgctcgttttcaatactacctctct

SEQ ID NO: 17 - DNA, 26 bp, Artificial Sequence
  Synthetic Polynucleotide
  misc_feature: TTN Probe
tccgcagctctagtggaagaaccacc

SEQ ID NO: 18 - DNA, 20 bp, Artificial Sequence
  Synthetic Polynucleotide
  misc_feature: P0 Forward
ctccaagcagatgcagcaga

SEQ ID NO: 19 - DNA, 20 bp, Artificial Sequence
  Synthetic Polynucleotide
  misc_feature: P0 Reverse
atagccttgcgcatcatggt

SEQ ID NO: 20 - DNA, 22 bp, Artificial Sequence
  Synthetic Polynucleotide
  misc_feature: P0 Probe
ccgtggtgctgatgggcaagaa

SEQ ID NO: 21 - RNA, 22 nt, Artificial Sequence
  Synthetic Polynucleotide
  misc_feature: miR208a
auaagacgagcaaaaagcuugu